(19) United States
(12) Patent Application Publication
(10) Pub. No.: US 2018/0070882 A1
(43) Pub. Date: Mar. 15, 2018

(54) METHOD, MEDICAL IMAGING APPARATUS AND MOBILE TERMINAL FOR PLANNING AND/OR CONTROLLING A MEDICAL IMAGING EXAMINATION

(71) Applicant: Siemens Healthcare GmbH, Erlangen (DE)

(72) Inventors: Rainer Schneider, Erlangen (DE); Dirk Franger, Erlangen (DE); Arne Hengerer, Moehrendorf (DE)

(73) Assignee: Siemens Healthcare GmbH, Erlangen (DE)

(21) Appl. No.: 15/698,057

(22) Filed: Sep. 7, 2017

(30) Foreign Application Priority Data
Sep. 9, 2016 (DE) 102016217204.2

(51) Int. Cl. A61B 5/00

(52) U.S. Cl. CPC A61B 5/721; A61B 5/0022; A61B 5/0002; A61B 5/7445; A61B 5/749; A61B 5/7475; A61B 5/6844

(57) ABSTRACT

In a method and apparatus for planning and/or controlling a medical imaging examination using a mobile terminal, a data connection is established between a medical imaging apparatus and the mobile terminal by a data network unit. Examination-specific data are transmitted to the mobile terminal. A command-relevant input is detected by at least one sensor of the mobile terminal. A planning command and/or a control command is generated in the mobile terminal on the basis of the detected signal and as a function of the examination-specific data. The planning command and/or control command is/are transmitted to a control computer of the medical imaging apparatus. The planning command and/or control command is/are executed by the control computer in order to plan and/or control the medical imaging examination.

### BACKGROUND OF THE INVENTION

### Field of the Invention

The present invention concerns a method for planning and/or controlling a medical imaging examination with the use of a mobile terminal. Furthermore, the present invention concerns a correspondingly configured mobile terminal and a medical imaging system that has a medical imaging apparatus and a mobile terminal.

### Description of the Prior Art

Particularly for interventional medical imaging examinations, it is important for operating personnel responsible for the interventional medical imaging examinations, especially a physician, to remain within the examination room during the interventional medical imaging procedure, in order to carry out and/or monitor the intervention on the patient.

However, monitoring and/or inspection of the medical imaging examination, particularly a magnetic resonance examination, has been possible only for operating personnel responsible for the medical imaging examination situated in a control room, which is separate from the examination room. A control computer with a user console is located within the control room, from which the monitoring and/or inspection of the medical imaging examination takes place. Thus, if inspection and/or monitoring is/are possible within the examination room, this takes place via a user interface. A user interface of this type is located at a fixed position within the installation site of the medical imaging apparatus, and can thus not always be operated from the position of a physician, for instance during an interventional medical imaging examination.

### SUMMARY OF THE INVENTION

An object of the present invention is to enable planning and/or control of a medical imaging examination from the examination room in a simple and time-saving manner.

The inventive method for planning and/or controlling a medical imaging examination with a mobile terminal, has the following method steps. A data connection is established between a medical imaging apparatus and the mobile terminal by a data network unit. Examination-specific data are transmitted to the mobile terminal. A command relevant input is detected by a sensor of the mobile terminal. A planning command and/or a control command is/are generated at the mobile terminal on the basis of the detected signal and as a function of the examination-specific data. The planning command and/or control command is/are transmitted to a control computer of the medical imaging apparatus. The planning command and/or control command is/are executed by the control computer in order to plan and/or control the medical imaging examination.

The medical imaging examination is preferably carried out by a medical imaging apparatus. For the medical imaging examination, the medical imaging apparatus, in particular a medical data acquisition scanner of the medical imaging apparatus, is activated accordingly by the control computer.

For this purpose, the scanner of the medical imaging apparatus is situated within an examination room. If the medical imaging apparatus is a magnetic resonance apparatus, the scanner is a magnet unit that includes a basic field magnet, a gradient coil arrangement and a radio frequency antenna, for instance. If the medical imaging apparatus is a computed tomography apparatus, the scanner then includes a rotating x-ray detector unit, for instance. For this purpose it is necessary for the examination room to be externally and internally shielded in order to shield interferences that can influence the medical imaging examination, for instance a magnetic resonance examination, and that may also be caused by the medical imaging apparatus. For instance, in an embodiment of the medical imaging apparatus as a magnetic resonance apparatus, the examination room is shielded both with respect to magnetic fields and electromagnetic radiation, in particular radio frequency radiation.

The control computer and further control components of the medical imaging apparatus for controlling the scanner, and an operator console of the medical imaging apparatus, are situated within a control room. The control room is shielded with respect to electromagnetic radiation from the examination room. For instance, in the embodiment of the medical imaging apparatus as a magnetic resonance apparatus, the control room is shielded with respect to the basic magnetic field generated by the magnet unit, and against electromagnetic radiation, in particular radio frequency radiation.

The mobile terminal is a mobile terminal that is compatible with the medical imaging apparatus and that can be located within the examination room. The mobile terminal can be shielded, such as in the embodiment of the medical imaging apparatus as a magnetic resonance apparatus, with respect to the magnetic field generated by the magnet unit and against electromagnetic radiation, in particular radio frequency radiation. Moreover, the mobile terminal can also be shielded so that the scanner of the medical imaging apparatus, and thus the medical imaging examination, are not influenced by the mobile terminal.

In such cases, the medical imaging apparatus can be any medical imaging apparatus that is considered to be appropriate by those skilled in the art, such as a computed tomography apparatus, a PET apparatus (positron emission tomography apparatus), a magnetic resonance apparatus, an ultrasound apparatus, a C-arm apparatus, etc.

As used herein, a data network unit means a unit that enables a communication between individual electronic units, in particular various technical, primarily independent electronic units, such as a computer, but also between sensors and/or actuators and/or agents and/or other radio technology components, etc. To this end, the individual electronic units are combined with and/or connected to one another. In such cases the data network unit, or also to some extent the individual electronic units, can be designed as, or to include, a router, in particular a network router for instance.

In such cases, a data connection by the data network unit is preferably cable-free (wireless). The data connection may be, for example, a WLAN connection, a UMTS connection, a GSM connection, a Bluetooth connection, etc. The planning command and/or the control command is/are preferably transmitted by the data connection of the data network unit.

In such cases the examination-specific data can be current image data, which are determined from currently acquired raw data. Moreover, the examination-specific data can be current planning data such as parameter values of the medical imaging examination that are to be set. Furthermore, the examination-specific data may be patient-related data. Further examination-specific data that appear reasonable to those skilled in the art are possible.

The (at least one) sensor of the mobile terminal is preferably built into the mobile terminal and arranged within the mobile terminal. The at least one sensor is preferably formed by a sensor already installed as standard in the mobile terminal, so that a particularly simple and cost-effective mobile terminal can be used. The mobile terminal is a portable mobile terminal. In such cases the portable mobile terminal is preferably designed such that it can be carried in one hand by a user. Operation of the mobile terminal by the user using just one hand is made possible in this way.

The control computer of the medical imaging apparatus is preferably situated within the control room. In order to execute the planning command and/or the control command, the control computer has software and/or computer programs that is executed by a processor of the control computer. The software and/or computer programs can be stored in a memory of the control computer or in an external storage medium.

The invention enables a user, for instance a physician responsible for an interventional medical imaging examination, to plan and/or control a medical imaging examination with the mobile terminal from the examination room in a simple and time-saving manner. Operating personnel responsible for the medical imaging examination then no longer have to leave the examination room and visit a control room in order to plan and/or control the medical imaging examination. Instead the preparation, and the planning and/or control of the medical imaging examination, in particular an interventional medical imaging examination, can be monitored and supervised directly from the examination room.

Moreover, it is possible to plan and/or monitor the medical imaging examination and/or the medical imaging apparatus from a remote operator location. For instance, a second physician can monitor the medical imaging examination with the mobile terminal and/or execute settings, in particular changes to control parameters, for the medical imaging examination. In such cases the second physician or operator can be located in a building which is separate from the building in which the medical imaging apparatus is installed. The second physician or operator may even be in another city or country.

The mobile terminal in accordance with the invention can be a tablet PC or a smart phone, as a result of which a particularly compact mobile terminal can be achieved. Moreover, the operation of the mobile terminal can be made simple, such as operation using just one hand of the user, so that the other hand of the user remains available for monitoring and/or carrying out the intervention during the medical imaging examination.

In another embodiment of the invention, the (at least one) sensor of the mobile terminal is a motion sensor and/or an orientation sensor and/or a sensor for detecting gestures. The motion sensor is preferably designed to detect a movement, in particular a translatory motion, of the mobile terminal. The orientation sensor is preferably designed to detect a rotary motion of the mobile terminal. The sensor for detecting gestures is preferably designed to detect user gestures on a touch-sensitive surface, in particular a touch display or a touch screen, of the mobile terminal. In such cases the sensor for detecting gestures may be a touch-sensitive surface for detecting gestures. Alternatively or in addition, the sensor for detecting gestures can also have a camera and/or further units that appear reasonable to those skilled in the art.

A signal for a control command and/or a planning command can be detected particularly easily by the (at least one) sensor, or by multiple sensors. In particular, a gesture for a control command and/or a planning command, which is intuitive to the user, can be used in order to make an input or planning gestures and/or control gestures. Here a detected rotation of the mobile terminal can result in a control command and/or planning command for instance, which brings about a turning and/or rotation of an orientation of a slice to be measured in the control parameters of an examination protocol. Furthermore, a detected translatory motion of the mobile terminal can result in a control command for instance, which effects a changeover of slices in a display in the image data or also effects a translatory motion of a component of the medical imaging apparatus. Moreover, a control command and/or planning command which effect(s) a zoom-in into the image data can be initiated by a control gesture, by the user marking a small area by means of his or her fingers and drawing this area outward in all directions on the surface. Moreover, control commands and/or planning commands that bring about a change in the rotation parameters and/or translation parameters of an examination protocol can also be effected in the control parameters by a gesture made by the user.

In this way, simple operation of the mobile terminal is achieved for a user in order to control the medical imaging apparatus for a medical imaging examination. Moreover, the planning commands and/or control commands can be entered even when the operator is wearing gloves on his or her hands; this can be especially advantageous during an interventional procedure monitored by medical imaging.

A rotary motion of the mobile terminal can be detected by the orientation sensor and/or the motion sensor, and a planning command and/or a control command can be generated for a corresponding rotary motion of at least one component of the medical imaging apparatus on the basis of the detected rotary motion of the mobile terminal. For instance, a planning command and/or control command can be generated for a turning and/or rotation of a robot arm of a C-arm apparatus on the basis of the detected rotary motion. This enables the position of a robot arm of the C-arm apparatus to be changed particularly easily. Moreover, a planning command and/or control command can also be generated for a rotation for a further, rotatable and/or turnable component of the medical imaging apparatus. This also enables parameter values, in particular turning parameters and/or a rotation parameter, to be set in a simple and time-saving manner.

Alternatively or in addition, a rotary motion of the mobile terminal can be detected by the orientation sensor and/or the motion sensor. On the basis of the detected rotary motion of the mobile terminal, a planning command and/or a control command is/are generated for a corresponding change in a turning parameter and/or rotation parameter of the medical imaging examination and/or the detected medical image data. In this way a planning command and/or control command can be generated for a change in a turning parameter and/or a rotation parameter for an orientation and/or rotation of a slice to be measured in the control parameters on the basis of the detected rotary motion. This enables control parameters, in particular turning parameters and/or a rotation parameter, of a measuring protocol, to be set in a simple and time-saving manner.

As used herein, a control parameter means a protocol-specific control parameter, which can be adjusted and/or set by a user prior to a medical imaging examination on a patient. Typically control parameters are selected as a function of the examination object and/or a clinical question, in particular as a function of the reason for the examination by the medical imaging apparatus. One set of control parameters, on the basis of which set the medical imaging apparatus can be activated and image data can be generated, can be referred to as a protocol. The protocol-specific control parameter can designate a slice thickness and/or a slice orientation, etc. for a magnetic resonance examination, for instance. For example, the user can influence the image quality by skillfully selecting and/or adjusting the individual control parameters of a protocol.

Furthermore, a translatory motion of the mobile terminal can be detected by the orientation sensor and/or the motion sensor, and a planning command and/or a control command can be generated for a corresponding translatory motion of at least one component of the medical imaging apparatus on the basis of the detected translatory motion of the mobile terminal. For instance, a planning command and/or control command can be generated for a movement of a patient bed of the medical imaging apparatus on the basis of the detected translatory motion. This enables a simple change in position of a patient bed and/or further components of the medical imaging apparatus that appear reasonable to those skilled in the art.

Alternatively or in addition, a translatory motion of the mobile terminal can be detected by the orientation sensor and/or motion sensor, and a planning command and/or a control command is/are generated for a corresponding change in a control parameter of the medical imaging examination and/or a display parameter of the detected medical image data on the basis of the detected translatory motion of the mobile terminal. This also enables control parameters of a measurement protocol to be set in a simple and time-saving manner. For instance, a command relating to browsing through different slices of the detected medical image data can be generated by the translatory motion.

The rotary motions and/or the translatory motions of the mobile terminal can result, for instance, from a rotary motion and/or translatory motion executed by an operator and transmitted onto the mobile terminal. By combining the two sensors, a user can also select an area in the detected image data which lies outside of the examination area, in particular in front of the examination area, or behind the examination area, or adjacent to the examination area, etc., for an extended analysis or diagnosis provided that these areas are included in the detected image data. In such cases a rotary motion and/or translatory motion can also executed by a gesture by the user and detected by the sensor for detecting a gesture.

In a further embodiment of the invention, a gesture by the user of the mobile terminal is detected by the sensor for detecting gestures, and on the basis of the detected gesture, a planning command and/or a control command is/are generated in order to control at least one component of the medical imaging apparatus. Here both translation parameters and/or rotation parameters can likewise be entered by a user by making a gesture. For instance, a corresponding control command for activation is enabled by a detected rotary motion and/or a translatory motion of a finger of the user in order to execute a rotary motion and/or translatory motion of a component of the medical imaging apparatus. This enables control signals to be detected particularly easily for the medical imaging examination.

Alternatively or in addition, a gesture by the user of the mobile terminal can be detected by the sensor for detecting gestures, and on the basis of the detected gesture, a planning command and/or a control command is/are generated for a corresponding change in a control parameter of the medical imaging examination and/or a display parameter of the detected medical image data. For instance, a planning command and/or a control command for browsing through different slices in the detected image data can be generated here on the basis of a detected swipe gesture. Furthermore, a planning command and/or a control command can also be generated by a detected swipe gesture in order to displace image data shown on the display unit. Moreover, rotation parameters and/or translation parameters can also be determined by a gesture by the user. Furthermore, a planning command and/or a control command for zooming in on a section in the image data can be generated by means of a control gesture for instance, by the user marking a small area using his or her fingers and drawing this area outward in all directions on the surface. Furthermore, a planning command and/or a control command for shrinking a section in the image data can be generated on the basis of a control gesture, wherein the user executes the control gesture by means of his fingers for instance and marks an area and shrinks this area by drawing his fingers together. Moreover, further control parameters and/or planning parameters can also be input particularly easily by means of a gesture.

In an alternative embodiment of the invention, the examination-specific data include planning data of the medical imaging examination. This enables detection of changes to the planning data and/or planning commands by the at least one sensor of the mobile terminal.

The examination-specific data can be current measurement data, as a result of which information relating to a current measurement can be provided in order to control the current measurement of the medical imaging examination. This enables a direct change in control parameters of the current measurement based on the current measurement data. The current measurement data can be current image data for instance, which are transmitted directly to the mobile terminal after detection.

In another embodiment of the invention, the examination-specific data are transmitted in real time to the mobile terminal, and displayed in real time on the mobile terminal. In this way current measurement results of the medical imaging examination can be shown directly, and changes in the control parameters, based on the current measurement results, can be made directly. Moreover, this embodiment of the invention enables a direct and simple monitoring of the current measurement, even for a user who remains at a location that is remote from the medical imaging apparatus.

The control command and/or planning command can be transmitted in real time by the mobile terminal to the medical imaging apparatus and be executed there, in order to control a current medical imaging examination in real time. The control command transmitted in real time is preferably also executed in real time by the medical imaging apparatus. The control commands and/or planning commands are preferably transmitted in real time to the control computer of the medical imaging apparatus, and are executed in real time by the control computer. In this way, it is particularly easy for the user to have a direct influence on an ongoing or a currently executed medical imaging examination. The ongoing or currently executed medical imaging examination can be adjusted according to current measurement results for instance. It is also possible to respond particularly quickly to changes affecting the patient, etc. The planning command and/or the control command can be executed particularly advantageously during a currently executed medical imaging examination.

An inspection of currently executed changes can be achieved for a user if, after the planning command and/or the control command has/have been executed on the medical imaging apparatus, detected measurement data is subsequently transmitted in real time to the mobile terminal and is shown there.

Additional information relating to the medical imaging examination can be transmitted to the mobile terminal. The additional information preferably includes existing and/or available image data of the patient. In this way information relating to planning the medical imaging examination and/or to controlling a current measurement of the medical imaging examination can additionally be indicated and/or shown to a user. This also enables the medical imaging examination to be quickly adjusted to the additional information. The additional information transmitted to the mobile terminal is preferably displayed and/or shown to the user on the mobile terminal.

Furthermore, the invention encompasses a mobile terminal that has a sensor interface with at least one sensor, one control module and one transmission module. The mobile terminal is designed to be compatible with a medical imaging apparatus.

The sensor interface is designed to detect sensor signals for the method for planning and/or controlling the medical imaging examination of the medical imaging apparatus by means of the mobile terminal. Furthermore, the control module is designed to generate control commands and/or planning commands for the method for planning and/or controlling the medical imaging examination of the medical imaging apparatus by the mobile terminal. The transmission module is designed to transmit data between the medical imaging apparatus and the mobile terminal for a method for planning and/or controlling a medical imaging examination of a medical imaging apparatus by the mobile terminal. The transmission module is preferably designed here to continuously transmit data between the medical imaging apparatus and the mobile terminal. The transmission module is preferably designed to continuously transmit detected medical image data in real time between the medical imaging apparatus and the mobile terminal.

The mobile terminal can be used to carry out a simple and time-saving planning and/or control of a medical imaging examination for a user from the examination room. In particular, operating personnel responsible for the medical imaging examination then no longer have to leave the examination room and visit a control room in order to plan and/or control the medical imaging examination. Instead the preparation and also the planning and/or control of the medical imaging examination, in particular an interventional medical imaging examination, can be monitored and supervised directly from the examination room. Moreover, a planning and/or monitoring of the medical imaging apparatus is/are also possible from a remote operator location. For instance, a second physician can monitor the medical imaging examination and/or execute settings, in particular changes to control parameters, by means of the mobile terminal for the medical imaging examination, without having to be present at the location of the medical imaging examination.

In an embodiment of the mobile terminal, the mobile terminal is a tablet PC or a smart phone, so a particularly compact mobile terminal is achieved. Moreover, the operation of the mobile terminal can be made simple, such as operation using just one hand of a user, so that the other hand of the user can remain available for monitoring and/or carrying out the intervention during the medical imaging examination.

The at least one sensor of the mobile terminal can be a motion sensor, an orientation sensor and/or a sensor for detecting gestures. The motion sensor is preferably designed to detect a movement, in particular a translatory motion, of the mobile terminal. The orientation sensor is preferably designed to detect a rotary motion of the mobile terminal. The sensor for detecting gestures is preferably designed to detect user gestures on a touch-sensitive surface, in particular a touch display or a touch screen, of the mobile terminal. In such cases the sensor for detecting gestures may comprise a touch-sensitive surface for detecting gestures. Alternatively or in addition, the sensor for detecting gestures can also have a camera and/or further units that appear reasonable to those skilled in the art.

A signal for a control command and/or a planning command can be detected particularly easily by the at least one sensor and/or the number of sensors. A gesture for a control command and/or a planning command, which is intuitive to the user, can be detected in order to enter and/or detect planning gestures and/or control gestures. Moreover, this achieves the advantage of simple operation of the mobile terminal for a user. Furthermore, the planning commands and/or control commands can be entered when the operator is wearing gloves on his or her hands, which may be especially advantageous during an interventional medical imaging examination.

Furthermore, the invention encompasses a medical imaging system having a medical imaging apparatus and a mobile terminal as described above, wherein the medical imaging system is designed to execute the inventive method for planning and/or controlling a medical imaging examination of a medical imaging apparatus with a mobile terminal.

Such a medical imaging system enables a simple and time-saving planning and/or control of a medical imaging examination with the mobile terminal from the examination room. In particular, operating personnel responsible for the medical imaging examination then no longer have to leave the examination room and visit a control room in order to plan and/or control the medical imaging examination. Instead, the preparation and also the planning and/or control of the medical imaging examination, such as an interventional medical imaging examination, can be monitored and supervised directly from the examination room. Moreover, planning and/or monitoring of the medical imaging apparatus is/are also possible from a remote operator location. For instance, a second physician can monitor the medical imaging examination and/or execute settings by the mobile terminal, without having to be present at the location of the medical imaging examination.

The advantages of the inventive medical imaging apparatus correspond to the advantages of the inventive mobile terminal and/or the advantages of the inventive method for planning and/or controlling a medical imaging examination by means of a mobile terminal, as explained in detail above. Features, advantages and alternative embodiments noted above are applicable to the other aspects of the invention.

### BRIEF DESCRIPTION OF THE DRAWINGS

FIG. 1 shows an inventive medical imaging system with a medical imaging apparatus and a mobile terminal.

FIG. 2 shows an inventive method for planning and/or controlling a medical imaging examination by means of a mobile terminal.

### DESCRIPTION OF THE PREFERRED EMBODIMENTS

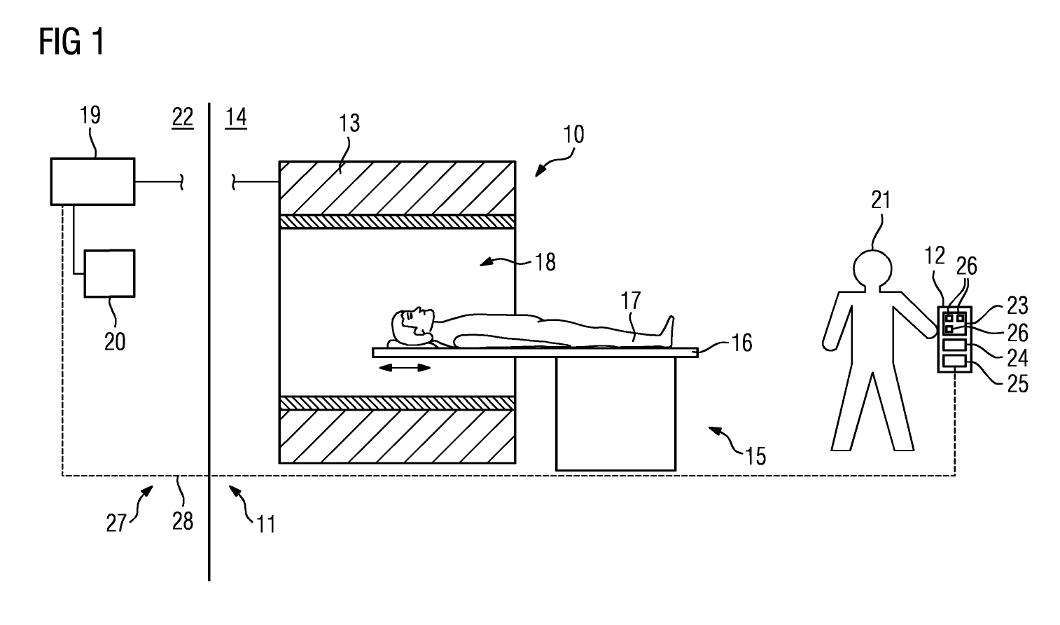

FIG. 1 is a schematic illustration of a medical imaging system 10 with a medical imaging apparatus 11 and a mobile terminal 12. The medical imaging apparatus 11 has a scanner 13 situated within an examination room 14. If the medical imaging apparatus 11 is a magnetic resonance apparatus, the scanner 13 is formed by a magnet unit that has a basic field magnet, a gradient coil arrangement, and a radio frequency antenna. If the medical imaging apparatus 11 is a C-arm apparatus, the scanner 13 is an x-ray detector. If the medical imaging apparatus 11 is a computed tomography apparatus, the scanner 13 is a rotating x-ray detector unit. Other embodiments of the medical imaging apparatus 11 that appear reasonable to those skilled in the art are also possible.

The medical imaging apparatus 11 has a patient support 15 with a patient bed 16, on which a patient 17 is supported for the medical imaging examination. The patient bed 16 is movably mounted within a patient receiving area 18 of the scanner 13. The patient receiving area 18 is surrounded at least partially by the scanner 13 in order to detect medical image data during the medical imaging examination. The patient support 15, together with the patient receiving area 18, is arranged within the examination room 14.

The medical imaging apparatus 11 has a control computer 19 and a user interface 20. The control computer 19 is designed to control the medical imaging apparatus 11. A user 21, in particular operating personnel responsible for the medical imaging examination, uses the user interface 20 to make inputs or obtain information via a display unit of the user interface 20. The control computer 19 and the user interface 20 are situated within a control room 22.

The control room 22 is shielded from the examination room 14 with respect to electromagnetic radiation. In the embodiment of the medical imaging apparatus 11 as a magnetic resonance apparatus, the control room 22 is shielded with respect to magnetic fields generated by the scanner 13, and against electromagnetic radiation, in particular radio frequency radiation, for instance. The examination room 22 is externally and internally shielded in order to shield interferences that can influence the medical imaging examination, for instance a magnetic resonance examination, as well as disturbing influences caused by the medical imaging apparatus 11. For instance, if the medical imaging apparatus 11 is a magnetic resonance apparatus, the examination room 22 is shielded both with respect to magnetic fields and against electromagnetic radiation, in particular radio frequency radiation.

The medical imaging system 10 is designed to execute the inventive method for planning and/or controlling a medical imaging examination by means of the mobile terminal 12. The mobile terminal 12 of the medical imaging system 10, which is formed by a smart phone or a tablet PC for instance, to this end has a sensor interface 23 with at least one sensor 26, a control module 24 and a transmission module 25.

The sensor 26 of the mobile terminal 12 can be a motion sensor for detecting a movement of the mobile terminal 12. Alternatively or in addition, the sensor 26 of the mobile terminal 12 can be an orientation sensor for detecting an orientation and/or position of the mobile terminal 12. Alternatively or in addition, the sensor 26 of the mobile terminal 12 can be a sensor for detecting gestures of a user 21, in particular of medical operating personnel, such as a physician. In such cases the sensor 26 for detecting gestures can have a touch-sensitive surface for detecting the gestures. Alternatively or in addition, the sensor 26 for detecting gestures can have a camera and/or further units that appear reasonable to those skilled in the art. In the present exemplary embodiment, the sensor interface 23 has both a sensor 26 embodied as a motion sensor, and a sensor 26 embodied as an orientation sensor, and a sensor 26 embodied as a sensor for detecting gestures. In principle, the number of sensors 26 of the sensor interface 23 can vary, or different embodiments of the individual sensors 26 can be used.

The sensor interface 23 is designed to detect sensor signals for the method for planning and/or controlling the medical imaging examination of the medical imaging apparatus 11 by means of the mobile terminal 12. The control module 24 is configured to generate control commands and/or planning commands for the method for planning and/or controlling the medical imaging examination of the medical imaging apparatus 11 by means of the mobile terminal 12.

The transmission module 25 is designed to transmit data between the medical imaging apparatus 11 and the mobile terminal 12 for a method for planning and/or controlling a medical imaging examination of a medical imaging apparatus 11 by means of the mobile terminal 12. The transmission module 25 is preferably designed to continuously transmit data between the medical imaging apparatus 11 and the mobile terminal 12. The transmission module 25 is preferably designed to continuously transmit detected medical image data in real time between the medical imaging apparatus 11 and the mobile terminal 12.

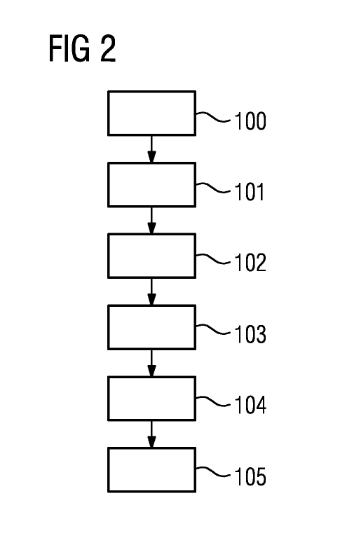

FIG. 2 shows the inventive method for planning and/or controlling a medical imaging examination of the medical imaging apparatus 11 with the mobile terminal 12. When executing the method, the mobile terminal 12 can be located at any location. For instance, it is also possible for the mobile terminal 12 to be located within the examination room 14. Moreover, a planning and/or monitoring of a medical imaging examination of the medical imaging apparatus 11 by means of the mobile terminal 12 is/are also possible from a remote operator location, for instance with a physician in another hospital.

In a first method step 100 of the method for planning and/or controlling the medical imaging examination of the medical imaging apparatus 11 by the mobile terminal 12, a data connection 28 is established between the medical imaging apparatus 11 and the mobile terminal 12 by a data network unit 27 of the medical imaging system 10. The data network unit 27 enables communication between individual, in particular various technical, primarily independent electronic units, such as for instance computers, but also between sensors, actuators, agents and other radio technology components etc. To this end, the individual electronic units are combined with and/or connected to one another. Here the data network unit 27, or in some cases also the individual electronic units, can include a router, in particular a network router, for instance. A data connection 28 is established by the data network unit 27 between the transmission module 25 of the mobile terminal 12 and the control unit 19 of the medical imaging apparatus 11. A data connection 28 by the data network unit 27 can preferably be cable-free (wireless). The data connection 28 may be a WLAN connection, a UMTS connection, a GSM connection, a Bluetooth connection etc. for instance.

If the mobile terminal 12 is situated within the examination room 14, data are exchanged by the data network unit 27 between the control room 22 and the examination room 14, in particular between the control computer 19 and the mobile terminal 12.

In a subsequent method step 101 examination-specific data are transmitted to the mobile terminal 12. The examination-specific data are preferably transmitted from the control unit 19 by the data connection 28 of the data network unit 27 to the mobile terminal 12.

The examination-specific data can in such cases include planning data of the medical imaging examination, such as parameter values of control parameters of the medical imaging examination which are to be set. Moreover, the examination-specific data can include current measurement data. In such cases the current measurement data can also be transmitted in real time to the mobile terminal 12 and displayed in real time on the mobile terminal 12. Furthermore, the examination-specific data can also include further examination-specific data that appear reasonable to those skilled in the art.

Aside from transmitting examination-specific data, in the method step 101 additional information relating to the medical imaging examination can also be transmitted to the mobile terminal 12 by the data connection 28 of the data network unit 27. The additional information relating to the medical imaging examination can in such cases be existing image data of the patient 17 from a previous or earlier imaging examination.

In a further method step 102, a signal is detected by the sensor interface 23, in particular by the at least one sensor 26, of the mobile terminal 12. The detected signal is preferably formed by a planning signal and/or a control signal. The signal, in particular the planning signal and/or the control signal, can in such cases be detected from a movement of the mobile terminal 12 by means of the motion sensor of the mobile terminal 12. Moreover, the signal, in particular the planning signal and/or the control signal, can be detected from a change in an orientation of the mobile terminal 12 by means of the orientation sensor of the mobile terminal 12. Furthermore, the signal, in particular the planning signal and/or the control signal, can also be detected from a gesture executed by the user 21 and/or operator of the mobile terminal 12 by the sensor of the mobile terminal 12 for detecting gestures.

In a further method step 103, a planning command and/or a control command is/are generated on the basis of the detected signal, in particular of the detected planning signal and/or of the detected control signal, and as a function of the examination-specific data. The planning command and/or the control command is/are generated by the control module 24 of the mobile terminal 12.

For instance, in the method step 102 of detecting a signal by means of the at least one sensor 26 a rotary motion of the mobile terminal 12 can be detected here by means of the orientation sensor and/or the motion sensor. In the further method step 103 a planning command and/or a control command can then be generated for a corresponding rotary motion of at least one component of the medical imaging apparatus 11 by means of the control module 24 on the basis of the detected rotary motion of the mobile terminal 12. The rotary motion of a component of the medical imaging apparatus can be a rotary motion of a robot arm of a C-arm apparatus for instance and/or further rotatable components that appear reasonable to those skilled in the art.

Alternatively or in addition, in the further method step 104 of generating a planning command and/or a control command, a planning command and/or a control command for a corresponding change in a turning parameter and/or rotation parameter of the medical imaging examination can also be generated by means of the control module 24 on the basis of the detected rotary motion of the mobile terminal 12. For instance, a detected rotation of the mobile terminal 12 can bring about a turning and/or a rotation of an orientation of a slice to be measured in the control parameters.

Alternatively or in addition, in the further method step 104 of generating a planning command and/or a control command, a planning command and/or a control command for a corresponding change in a turning parameter and/or rotation parameter of the detected medical image data can also be generated by means of the control module 24 on the basis of the detected rotary motion of the mobile terminal 12. In this way an orientation of a display and/or a view of the detected medical image data can be changed, for instance.

Furthermore, a translatory motion of the mobile terminal 12 can be detected by means of the orientation sensor and/or the motion sensor. In the further method step 104 of generating a planning command and/or a control command, a planning command and/or a control command can then be generated for a corresponding translatory motion of at least one component of the medical imaging apparatus 11 by the control module 24 on the basis of the detected translatory motion of the mobile terminal 12. For instance, a planning command and/or control command for a movement of the patient couch 16 of the medical imaging apparatus 11 can be generated here on the basis of the detected translatory motion.

Alternatively or in addition, in the further method step 104 of generating a planning command and/or a control command, a planning command and/or a control command can then also be generated for a corresponding change in a parameter value of a measurement parameter of the medical imaging examination and/or a display parameter of the detected medical image data by means of the control module 24 on the basis of the detected translatory motion of the mobile terminal 12. A detected translatory motion of the mobile terminal 12 can effect a changeover of the slices in a display in the image data, for instance.

Furthermore, a gesture by a user 21 of the mobile terminal 12 can be detected by means of the sensor for detecting gestures. In the further method step 104 of generating a planning command and/or a control command, a planning command and/or a control command can be generated for controlling at least one component of the medical imaging apparatus by means of the control module 24 on the basis of the detected gesture. Alternatively or in addition, in the further method step 104 of generating a planning command and/or a control command, a planning command and/or a control command can be generated for a corresponding change in a control parameter of the medical imaging examination by means of the control module 24 on the basis of the detected gesture. Alternatively or in addition, in the further method step 104 of generating a planning command and/or a control command, a planning command and/or a control command can be generated for a corresponding change in a display parameter of the detected medical image data by means of the control module 24 on the basis of the detected gesture.

For instance, a planning command and/or a control command for browsing through different slices in the detected image data can be generated on the basis of a detected swipe gesture. Furthermore, a planning command and/or a control command can also be generated by a detected swipe gesture in order to displace image data shown on the display unit. Moreover, rotation parameters and/or translation parameters can also be determined by means of a gesture by the user. For instance, a corresponding control command for activation can be triggered by means of a detected rotary motion and/or a translatory motion of a finger of the user in order to execute a rotary motion and/or translatory motion of a component of the medical imaging apparatus 11 and/or to change a rotation parameter and/or translation parameter.

Moreover, a control gesture can be used to generate a planning command and/or a control command for zooming in on a section in the image data for instance, by the user 21 marking a small area using his or her fingers and drawing this area outward in all directions on the surface. Furthermore, a planning command and/or a control command for shrinking a section in the image data can be generated on the basis of a control gesture, wherein the user 21 executes the control gesture by his or her fingers, for instance, and marks an area and shrinks this area by drawing his or her fingers together.

In a further method step 104, the planning command and/or the control command is/are transmitted from the mobile terminal 12 to the medical imaging apparatus 11, in particular to the control computer 19 of the medical imaging apparatus 11. The planning command and/or control command is/are preferably transmitted by means of the data connection 28 of the data network unit 27 between the transmission module 25 of the mobile terminal 12 and the control computer 19 of the medical imaging apparatus 11. The planning command and/or control command is/are particularly advantageous transmitted here in real time to the control computer 19 of the medical imaging apparatus 11, which is advantageous particularly with ongoing medical imaging examinations.

In a further method step 105, the planning command and/or control command is/are executed by means of the control unit 19 of the medical imaging apparatus 11 for planning and/or controlling the medical imaging examination. Advantageously, the planning command and/or the control command is/are executed here in real time by the control computer 19.

In the further method step 105 of executing the planning command and/or the control command, the planning command and/or the control command can be executed during a currently ongoing medical imaging examination, by the control computer 19 operating the medical imaging apparatus 11.

After the planning command and/or the control command has/have been executed by means of the control computer 19 on the medical imaging apparatus 11, the detected measurement data can then be transmitted in real time to the mobile terminal 12 by means of the data connection 28 of the data network unit 27 and displayed and/or shown there to the user 21 of the mobile terminal 12. This enables immediate inspection of the settings in the planning commands and/or control commands for the medical imaging examination.

Although modifications and changes may be suggested by those skilled in the art, it is the intention of the Applicant to embody within the patent warranted hereon all changes and modifications as reasonably and properly come within the scope of the Applicant's contribution to the art.

1. A method for planning and/or controlling a medical imaging examination using a mobile terminal, said method comprising:
establishing a data connection between a medical imaging apparatus and a mobile terminal via a data network;
transmitting examination-specific data to the mobile terminal that is specific for a medical imaging examination to be implemented by the medical imaging apparatus;
detecting a command relevant input with at least one sensor of the mobile terminal;
in said mobile terminal, generating a planning command and/or a control command based on the detected command relevant input and dependent on the examination-specific data;
transmitting the planning command and/or the control command from the mobile terminal to the medical imaging apparatus; and
at the medical imaging apparatus, executing the planning command and/or the control command via the control computer, in order to plan and/or control the medical imaging examination.

2. A method as claimed in claim 1 comprising using a tablet PC or a smart phone as said mobile terminal.

3. A method as claimed in claim 1 comprising selecting said sensor of said mobile terminal from the group consisting of a motion sensor, an orientation sensor, and a gesture-detecting sensor.

4. A method as claimed in claim 1 comprising selecting said sensor of the mobile terminal from the group consisting of an orientation sensor and a motion sensor, and, with said sensor of said mobile terminal, detecting, as said command relevant input, a rotary motion of the mobile terminal, and generating said planning command and/or said control command as a command to execute a corresponding rotary motion by at least one component of the medical imaging apparatus.

5. A method as claimed in claim 1 wherein said medical imaging examination comprises at least one parameter selected from the group consisting of a turning parameter and a rotation parameter, and wherein execution of said medical imaging examination produces medical image data, and wherein said sensor of said mobile terminal is selected from the group consisting of an orientation sensor and a motion sensor, and wherein said sensor of said mobile terminal detects a rotary motion, as said command relevant input, and wherein said planning command and/or said control command produces a corresponding rotary change in said at least one parameter of said medical imaging examination and/or of said medical image data.

6. A method as claimed in claim 1 comprising selecting said sensor of the mobile terminal from the group consisting of an orientation sensor and a motion sensor, and, with said sensor of said mobile terminal, detecting, as said command relevant input, a translatory motion of the mobile terminal, and generating said planning command and/or said control command as a command to execute a corresponding translatory motion by at least one component of the medical imaging apparatus.

7. A method as claimed in claim 1 wherein said medical imaging examination comprises at least one parameter selected from the group consisting of a turning parameter and a rotation parameter, and wherein execution of said medical imaging examination produces medical image data, and wherein said sensor of said mobile terminal is selected from the group consisting of an orientation sensor and a motion sensor, and wherein said sensor of said mobile terminal detects a translatory motion, as said command relevant input, and wherein said planning command and/or said control command produces a corresponding translatory change in said at least one parameter of said medical imaging examination and/or of said medical image data.

8. A method as claimed in claim 1 wherein said sensor of said mobile terminal is a gesture-detecting sensor, and wherein said planning command and/or said control command controls at least one component of the medical imaging apparatus dependent on a gesture, as said command relevant input, detected by said gesture-detecting sensor.

9. A method as claimed in claim 1 wherein said sensor of said mobile terminal is a gesture-detecting sensor, and wherein said medical imaging examination has at least one control parameter, and produces medical imaging data that is displayed according to at least one display parameter, and wherein said gesture-detecting sensor detects a gesture, as said command relevant input, made by a user of the mobile terminal and wherein said planning command and/or said control command produces a change, corresponding to said gesture, in said at least one control parameter and/or said at least one display parameter.

10. A method as claimed in claim 1 wherein said examination-specific data comprise planning data for said medical imaging examination.

11. A method as claimed in claim 1 wherein said examination-specific data comprise current measurement data acquired during an ongoing medical imaging examination.

12. A method as claimed in claim 1 comprising transmitting said examination-specific data to said mobile terminal in real time, and displaying said examination-specific data in real time at said mobile terminal.

13. A method as claimed in claim 1 comprising transmitting said control command and/or planning command from the mobile terminal to the medical imaging apparatus in real time, and executing said control command and/or said planning command at said medical imaging apparatus in real time.

14. A method as claimed in claim 13 comprising executing the planning command and/or the control command during a current, ongoing medical imaging examination.

15. A method as claimed in claim 14 wherein said current, ongoing medical imaging examination produces measurement data, and, after the planning command and/or the control command has been executed by the medical imaging apparatus, transmitting the detected measurement data in real time to the mobile terminal, and displaying the measurement data at the mobile terminal.

16. A method as claimed in claim 1 comprising transmitting additional information relating to the medical imaging examination to the mobile terminal.

17. A method as claimed in claim 16 comprising transmitting, as said additional information, existing image data of a patient for whom the medical imaging examination is to be conducted.

18. A mobile terminal for planning and/or controlling a medical imaging examination to be executed by a medical imaging apparatus, said mobile terminal comprising:
a data interface configured to establish a connection between a medical imaging apparatus and a mobile terminal via a data network;
said data interface being configured to receive examination-specific data transmitted to the mobile terminal that is specific for a medical imaging examination to be implemented by the medical imaging apparatus;
at least one sensor that detects a command-relevant input;
a command generator configured to generate a planning command and/or a control command based on the detected command relevant input and dependent on the examination-specific data; and
a transmitter that transmits the planning command and/or the control command from the mobile terminal to the medical imaging apparatus.

19. A mobile terminal as claimed in claim 18 wherein said transmission between said mobile terminal and said medical imaging apparatus occurs continuously.

20. A mobile terminal as claimed in claim 18 wherein said mobile terminal is a tablet PC or a smart phone.

21. A mobile terminal as claimed in claim 18 wherein said sensor is a sensor selected from the group consisting of a motion sensor, an orientation sensor, and a gesture-detecting sensor.

22. A medical imaging system comprising:
a medical imaging apparatus;
a mobile terminal comprising a data interface configured to establish a connection between a medical imaging apparatus and a mobile terminal via a data network, said data interface being configured to receive examination-specific data transmitted to the mobile terminal that is specific for a medical imaging examination to be implemented by the medical imaging apparatus, at least one sensor that detects a command-relevant input, a command generator configured to generate a planning command and/or a control command based on the detected command relevant input and dependent on the examination-specific data, a transmitter that transmits the planning command and/or the control command from the mobile terminal to the medical imaging apparatus; and
a control computer configured to operate the medical imaging apparatus in order to execute the planning command and/or the control command so as to plan and/or control the medical imaging examination.
